(19) United States
(12) Patent Application Publication
(10) Pub. No.: US 2007/0020266 A1
(43) Pub. Date: Jan. 25, 2007

(54) Gpr54 receptor agonist and antagonist useful for the treatment of gonadotropin related diseases

(75) Inventors: Nicolas De Roux, BOIS-COLOMBES (FR); Emmanuelle Genin, Paris (FR); Edwin Milgrom, Seaux (FR); Jean-Louis Chaussain, Paris (FR); Jean-Claude Carel, Neuilly Sur Seine (FR)

Correspondence Address: STATTLER, JOHANSEN, AND ADELI LLP, 1875 CENTURY PARK EAST SUITE 1360, LOS ANGELES, CA, 90067, US

(73) Assignees: INSTITU NATIONAL DE LA SANTE ET DE LA RECHERCHE MEDICALE, PARIS-CEDEX 13 (FR); UNIVERSITY RENE DESCARTES PARIS V, ORSAY-CEDEX (FR); UNIVERSITY PARIS-SUD XI, ORSAY-CEDEX (FR)

(21) Appl. No.: 10/552,028

(22) PCT Filed: Apr. 2, 2004
(86) PCT No.: PCT/EP04/04132

§ 371 Date: Oct. 2, 2005

(30) Foreign Application Priority Data
Apr. 2, 2003 (EP) 0329083.1

(51) Int. Cl. A61K 39/395; C07K 16/28; C07K 16/26

(52) U.S. Cl. 424/145.1; 514/12; 530/388.24; 530/388.22

(57) ABSTRACT

The invention provides an agonist or antagonist of the GPR54 receptor for its use for treating a gonadotropin related disorder, a ligand of the GPR54 receptor for its use for diagnosing a subject's gonadotropin abnormality; a method for screening a compound that affect the gonadotropic axis comprising the step of assaying the compound in the presence of a GPR54 receptor and novel proteins useful in the above.

### FIELD OF THE INVENTION

The invention relates to GPR54 receptor agonist and antagonist that are useful for the treatment of gonadotropin related diseases, as well as in the diagnostic field. These compounds will find application in many pathologies, known to be dependant upon GnRH or LH/FSH secretion.

### BACKGROUND OF THE INVENTION

The integrity of the gonadotropic axis leads to normal sexual differentiation during fetal life, normal puberty and therefore to normal fertility. Genetic defects leading to isolated impuberism and infertility have been described in genes encoding for known proteins as GnRH receptor, gonadotropins, gonadotropin receptors and steroidogenic enzymes.

Hypogonadisms related to deficiency of GnRH or LH/FSH synthesis are called hypogonadotropic hypogonadism. Cases of congenital isolated hypogonadotropic hypogonadism are classified into 2 categories: those associated with anosmia (Kallmann syndrome) and those apparently isolated. Mutations and deletions of the KAL-1 gene have been observed in many cases of the X-linked form of Kallmann syndrome. Cases of hypogonadotropic hypogonadism without anosmia were considered as idiopathic until the recent description of mutations of the GnRH receptor gene.

Inhibition or activation of the gonadotropic axis are useful schemes for the treatment of hormones-related diseases, such as gonadotropin deficiency, precocious puberty, and some types of cancer (e.g. prostate and breast cancers) and useful to manage in-vitro fecundation. For the time being, only the GnRH receptor is known to play a role in regulating LH and FSH secretion, although it is possible that GnRH receptor may itself has regulation effects in cancer independent of LH and FSH.

GPR54 was initially described as an orphan receptor homolog to galanin receptor. Recently, a ligand acting on GPR54 bas been described. Katani et al as well as Ohtaki et al analysed placental extracts for peptides activating GPR54. Muir et al used a library of 1500 putative ligands. The best agonists displayed a similarity to a 54 amino acids peptide derived from the KiSS-1 protein (also found were peptides of 14, 13 and even fewer amino acids). This peptide corresponds to the predicted proteolytic processing of KiSS-1 at dibasic and dibasic/amidation sites, and have been named Kisspeptins. GPR54 stimulation by this 54 amino acid peptide results in the activation of phospholipase C by coupling to a Gq protein. It was also determined that GPR54 was mainly present in pituitary and placenta, and that Kisspeptins are high affinity agonists of the GPR54 receptor. Kotani et al. concludes that tissue distribution suggested that GPR54 might be implicated in various hormonal functions, a hypothesis supported by the demonstration that KISS-1 derived peptides stimulate oxytocin release in rats.

Human and rat GPR54 genes have been fully disclosed in many publications, referred to in the above Kotani, Ohtaki and Muir publications. GPR54 gene is formed of 5 exons. GPR54 has also sometimes been named AXOR12 or SNORF11.

WO-A-2003003983 discloses a method of treating an abnormality which comprises administering to the subject an amount of a SNORF11 (GPR54) receptor agonist. Examples of such agonists that are given include KISS-1 peptide fragments. It is also indicated that the SNORF11 (GPR54) receptor may serve as a tool for designing drugs for treating various pathological conditions, including, cancers, sexual/reproductive disorders, benign prostatic hypertrophy. The sole example given relates to pain.

U.S. Pat. No. 2,002,0106766 discloses the rat AXOR12 gene sequence and potential uses of agonists and antagonists of the receptor. There is however no working example of any pathology in this document.

EP-A-1126028 (naming Ohtaki as an inventor) discloses GPR54 gene, encoded protein, ligands and potential uses, which include diagnostic and screening uses. Rat and human GPR54 proteins and coding sequences are disclosed in SEQ ID NO:1 and 5, respectively.

However, none of the above documents teaches or suggests the present invention.

### SUMMARY OF THE INVENTION

The invention shows that GPR54 is a new hormonal system playing an important and previously unsuspected role in the physiology of the gonadotropic axis.

Hence, the invention offers a further route for defining new pharmacological strategies to activate or inhibit the gonadotropic axis and investigating gonadotropic hormones related pathologies.

The invention thus provides an agonist or antagonist of the GPR54 receptor for its use for treating a gonadotropin related disorder.

In one embodiment, the GPR54 receptor is the protein shown in SEQ ID NO:2 or SEQ ID NO:3, or a partial (functional) protein thereof, or an ester, amide or salt thereof.

In another embodiment, the GPR54 receptor is the protein shown in SEQ ID NO:2 or SEQ ID NO:3, from amino-acids 247 to 398.

In another embodiment, the GPR54 receptor is the protein shown in SEQ ID NO:2 or SEQ ID NO:3, with the mutation L102P.

The agonist or antagonist of the invention is useful for its use for treating hypogonadotropic hypogonadism, LH and/or FSH related disorders, gonadotropin-estradiol/testosterone-dependent related cancers and/or gonadotropin related reproductive disorders.

The invention also provides a ligand of the GPR54 receptor for its use for diagnosing a subject's gonadotropin abnormality, such as hypogonadotropic hypogonadism.

In one embodiment, the ligand of the invention binds to the protein shown in SEQ ID NO:2 or SEQ ID NO:3, from amino-acids 247 to 398.

In another embodiment, the ligand of the invention binds to the protein shown in SEQ ID NO:2 or SEQ ID NO: 3, with the mutation L102P.

The invention also provides a method for screening a compound that affect the gonadotropic axis comprising the step of assaying the compound in the presence of a GPR54 receptor.

In one embodiment, the screening method of the invention aims at screening for a compound that affects (for example effects) the LH and/or FSH secretion.

In one embodiment, in the screening method of the invention, the GPR54 receptor is the protein shown in SEQ ID NO:2 or SEQ ID NO:3, or a partial protein thereof, or an ester, amide or salt thereof.

In another embodiment, in the screening method of the invention, the GPR54 receptor is the protein shown in SEQ ID NO:2 or SEQ ID NO:3, from amino-acids 247 to 398.

In another embodiment, in the screening method of the invention, the GPR54 receptor is the protein shown in SEQ ID NO:2 or SEQ ID NO:3, with the mutation L102P.

The invention provides novel proteins, i.e. the proteins shown in SEQ ID NO:2 or SEQ ID NO:3, from amino-acids 247 to 398 as well as the protein shown in SEQ ID NO:2 or SEQ ID NO:3, with the mutation L102P, together with antibodies specific to these.

The invention also provides an agonist or antagonist of the GPR54 receptor for its use as an addition to a treatment for the stimulation of ovulation by GnRH,

In another embodiment the agonist or antagonist of GPR54 positively modulates the GnRH effect on LH synthesis stimulation. In another embodiment the GPR54 protein is the protein shown in SEQ ID NO:2 or SEQ ID NO:3, or a partial protein thereof, or an ester, amide or salt thereof. Additionally GPR54 can be the protein shown in SEQ ID NO:2 or SEQ ID NO:3, from amino-acids 247 to 398 or the protein shown in SEQ ID NO:2 or SEQ ID NO:3, with the mutation L102P

In another embodiment the invention also provides a composition comprising GnRH and agonists or antagonists to GPR54. In another embodiment the ratio of the GnRH to the agonist or antagonist of GPR54 is in the range 10:1 to 1000:1 in Molar concentration. In another embodiment the agonist is the fragment 45-54 of Kiss-1.

### BRIEF DESCRIPTION OF THE FIGURES AND SEQUENCES

FIG. 1 shows the pedigrees of the two affected families.

FIG. 2 is a GnRH (100 μg/iv) test performed in the propositus of family 2

FIG. 3 is the amino acid sequence of human GPR54. Boxes highlight putative transmembrane domains. The site of the deletion observed in affected individuals is indicated by an arrow. The deleted protein sequence is in italics.

FIG. 4: Functional characterisation of the L102P mutated receptor. Wild type and mutated plasmids were transfected within HEK293T cells. Inositol Phosphate accumulation was measured 48 hours after the transfection. Transfection with an empty expression vector (pcDNA) was used as a control.

FIG. 5: Kiss-1 effects on LH secretion: Two male rat pituitaries were pooled within each perifused chambers (four chambers for each condition). A buffer was perifused within each chamber with a flow of 0.1 ml/mn. Samples were collected every 5 minutes. Stimulations were performed three times at 50 minutes interval. This figure shows the result from four different chambers (two chambers with GnRH alone being chambers 5 and 6 and two chambers with GnRH and Kiss-1 (fragment 45-54, being a functional truncation of Kiss-1) being chambers 9 and 10), Open boxes indicate the time of the agonists injection. No stimulation was observed for kiss-1 alone.

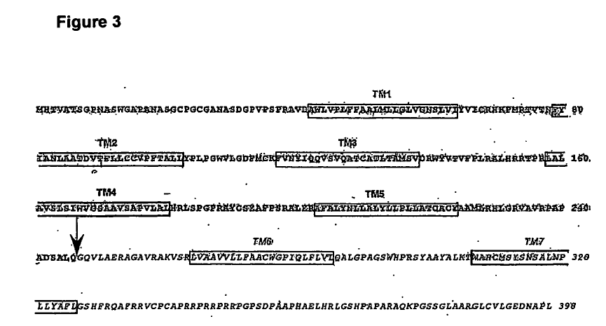

SEQ ID NO:1 is the sequence of human GPR54 (gene and encoded protein) while SEQ ID NO:2 is the sequence of the protein. SEQ ID NO:3 is another sequence for the protein of human GPR54 showing a polymorphic variation at position 364 according to which leucine is replaced by histidine (corresponding to the GPR54 shown in FIG. 3).

### DETAILED DESCRIPTION OF THE INVENTION

As indicated above, the inventors have found that GPR54 plays an important and previously unsuspected role in the physiology of the gonadotropic axis. The present invention describes a new genetic etiology for impuberism. It shows that alteration of GPR54 (KiSS-1 peptide receptor) plays an important and previously unsuspected role in the initiation of puberty. Therefore, loss of function of GPR54 leads to hypogonadotropic hypogonadism.

This was demonstrated by sequencing the GPR54 nucleotide sequence of affected patients, where the patients were suffering from hypogonadotropic hypogonadism (impuberism). GPR54 was chosen as candidate gene as it is localized in the region of interest defined by genome mapping in a very informative family. A homozygous deletion within intron 4 and exon 5 of the GPR54 gene was found in all affected siblings in one family. In a second family showing a recessive transmission, a loss of function homozygous point mutation of GPR54 was found within exon 1.

These findings will have useful applications in diagnostic and drug design, in pathologies that are related to GnRH or gonadotropin secretion.

The GPR54 receptor proteins and the like are useful, among other things: (1) for determination of an agonist or antagonist to the GPR54 receptor, where these agonist and antagonist compounds would be useful in gonadotropin-related diseases, (2) for screening of compounds (agonist, antagonist, etc.) that alter the binding property between GPR54 and a ligand, whereby the screened compound would then be useful for the treatment of gonadotropin-related diseases and (3) for determination of an agonist or antagonist to the GPR54 receptor where these agonist and antagonist compounds would be useful in assisted reproduction (4) for diagnosing gonadotropin-related diseases, as a genetic diagnostic agent or (5) for determination of a compound leading to perform dynamic hormonal tests of the gonadotropic axis during diagnosis procedure.

Gonadotropin-related diseases include those pathologies involving malfunction in the LH and/or FSH secretion, hypogonadotropic hypogonadism, precocious puberty, uterine leiomyomas), severe endometriosis, hyperandrogenism, menometrorrhagia, catamenial disorders and endometrial hyperplasia, and prostate and breast cancers known to be LH-dependent estradiol/testosterone-dependent disorders.

The administration of agonists or antagonists of GPR54 leading to the activation or inhibition of the gonadotropic axis, can be suitable for treating the gonadotropin-related diseases. Agonists or antagonists of GPR54 can be indeed useful for stimulating or inhibiting GnRH or LH/FSH synthesis.

Agonists of GPR54 are herein defined as compounds able to activate GPR54.

Technologies related to gene therapy are known to the skilled man. Said agonists can also consist in compounds that mimic the natural ligand of GPR54, such as kiss-1 peptide, kisspeptins, or derived peptides thereof. Suitable agonists to the receptor GPR54 can be specific or not, and can consist in natural or synthetic compounds. These agonists include but are not limited to Kiss-1 peptide itself, Kiss-1 peptide fragments, kisspeptins, or salt thereof, but can also include those described in WO-A-200300398, U.S. Pat. No. 2,002,0106766, and EP-A-1126028. Thus the invention proposes the use of an agonist of GPR54 for treating diseases related to a deficiency of GnRH or LH/FSH.

Antagonists of GPR54 are herein defined as a compounds able to inhibit the activation or the expression of GPR54.

Compounds capable of inhibiting the activation of GPR54 include in particular those able to interact with natural agonists of GPR54, such as kiss-1 peptide, to inhibit the binding of said agonists, or to inhibit the activation of GPR54 resulting from said binding. For instance, a inhibitor of activation of GPR54 can consist in an antibody directed to Kiss-1 peptide, or kisspeptins, which impedes the binding of said Kiss-1 peptide, or said kisspeptins to GPR54.

Inhibitors of the expression of GPR54 include for instance antisense oligonucleotides, or interfering RNAsi, or ribozymes, targeting the GPR54 gene.

Antisense nucleic acids that can inhibit replication or expression of the GPR54 receptor protein gene can inhibit RNA synthesis or the function of RNA, or can regulate/control the expression of the receptor protein gene via the interaction with RNAs associated with the receptor protein. Antisense nucleic acids are useful for regulating and controlling the expression of the receptor protein gene in vivo and in vitro, and are also useful for the treatment and diagnosis of the diseases described above.

Technologies related to such antisense RNAs and gene therapies are known to the skilled man.

Said agonists or antagonists can be administered by themselves, or mixed with suitable carriers or excipients.

Said agonists and antagonists can be further identified by the screening methods described hereinafter.

The screening methods of the invention can be carried out according to known methods. Those depicted in EP-A-1126028, WO-A-2003003983 and U.S. Pat. No. 2,002,0106766 are suitable.

The screening method may measure the binding of a candidate compound to the receptor, or to cells or membranes bearing the receptor, or a fusion protein thereof by means of a label directly or indirectly associated with the candidate compound. Alternatively, a screening method may involve measuring or, qualitatively or quantitatively, detecting the competition of binding of a candidate compound to the receptor with a labelled competitor (e.g., agonist or antagonist). Further, screening methods may test whether the candidate compound results in a signal generated by an agonist or antagonist of the receptor, using detection systems appropriate to cells bearing the receptor. Antagonists can be assayed in the presence of a known agonist and an effect on activation by the agonist by the presence of the candidate compound is observed. Further, screening methods may comprise the steps of mixing a candidate compound with a solution comprising a GPR54 receptor, to form a mixture, and measuring the activity in the mixture, and comparing to a control mixture which contains no candidate compound. Competitive binding using known peptide agonist such as the KISS peptides mentioned above is also suitable.

Assays techniques are known in the art and the skilled man may revert to publications to that effect, such as the mentioned patents, e.g. EP-A-1126028, WO-A-2003003983 and U.S. Pat. No. 2,002,0106766.

The GPR54 receptor of the present invention may be employed in conventional low capacity screening methods and also in high-throughput screening (HTS) formats.

Screening kits can then be manufactured using known techniques.

Once screened and identified, the useful compounds are conventionally used as pharmaceutical compositions.

The diagnostic methods may be carried out using the methods disclosed in EP-A-1126028. Notably, antibodies can be used, where the antibodies, monoclonal or polyclonal can be manufactured by publicly known methods.

Laboratory methods for preparing monoclonal antibodies are well known in the art. Monoclonal antibodies (mabs) may be prepared by immunizing purified mutated GPR54 protein into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. Phage display technology is also a useful technology for the production of mabs.

Antibodies according to the invention are designed to be specific to the mutated form of GPR54 protein, ie; that are capable of distinguishing between a mutated form of GPR54 protein and the wild-type GPR54 protein.

Especially, antibodies shall permit to identify the GPR54 protein lacking its transmembrane domains 6 and 7, or having the point mutation L102P.

Other ligands can be used, as long as they allow recognition the presence (or absence) of (part) of the GPR54 protein.

Diagnostic kits involving nucleic acids or protein assays can then be manufactured using known techniques.

The GPR54 protein useful in the present invention is one that has an amino sequence identical or substantially similar to the one depicted in SEQ ID NO:2 or SEQ ID NO:3. Preferably, the sequence includes an amino acid sequence having at least about 70% homology, preferably at least about 80% homology, more preferably at least about 90% homology, most preferably at least about 95% homology, to the protein sequence represented by either SEQ ID NO:2 or SEQ ID NO:3. Partial peptides can be used.

The instant invention is not limited to human GPRS4, but can be applied to any other mammals, including those useful in the agricultural field, it being understood that the GPR54 is the one corresponding to the mammal of interest.

Specific examples include the protein corresponding to the polypeptide from residue 247 to 398 of SEQ ID NO:2 (hereinafter deleted or truncated GPR54 protein) or the polypeptide shown in SEQ ID NO:2 with the mutation L102P (proline substituted for leucine) (hereinafter 102-mutated GPR54 protein) or the polypeptide shown in SEQ ID NO:3 (bearing a polymorphism such that leucine is replaced by histidine at position 364 compared to SEQ ID NO:2) or the polypeptide shown in SEQ ID NO:3 with the mutation L102P or the polypeptide from residue 247 to 398 of SEQ ID NO:3.

The receptor protein of the present invention which can be used may be a protein comprising (i) an amino acid sequence represented by SEQ ID NO:2 or SEQ ID NO:3, or the truncated or 102-deleted corresponding protein, in which one, two, or more amino acids (preferably 1 to 30 amino acids, more preferably 1 to 10 amino acids, most preferably 1 or 2 amino acids) are deleted; (ii) an amino acid sequence represented by SEQ ID NO:2 or SEQ ID NO:3, or the truncated or 102-deleted corresponding protein, to which one, two, or more amino acids (preferably 1 to 30 amino acids, more preferably 1 to 10 amino acids, most preferably 1 or 2 amino acids) are added; (iii) an amino acid sequence represented by SEQ ID NO:2 or SEQ ID NO:3, or the truncated or 102-deleted corresponding protein, in which one, two, or more amino acids (preferably 1 to 30 amino acids, more preferably 1 to 10 amino acids, and most preferably 1 or 2 amino acids) are substituted by other amino acids; and (iv) a combination of the above amino acid sequences.

The partial peptide of the GPR54 receptor protein of the present invention (hereinafter sometimes referred to as the partial peptide) may be any partial peptide, so long as it constitutes a part of the peptide portions of the receptor protein described above retaining binding properties. Examples of such partial peptides include site, which is exposed outside cell membranes among the receptor protein and retain the receptor binding activity or the transmembrane domains. These domains are identified in FIG. 3.

An example is a peptide containing a region which is analyzed to be an extracellular area (hydrophilic region or site) in a hydrophobic plotting analysis.

It is also possible to have partial peptides fused together.

The number of amino acids in the partial peptide of the present invention is at least 20 or more, preferably 50 or more, more preferably 100 or more, in terms of the constructive amino acid sequence of the GPR54 receptor protein described above.

Esters, amides or salts can also be used, as disclosed in EP-A-1126028.

The receptor protein of the present invention may be manufactured in accordance with a publicly known method for purification of a receptor protein from human or other mammalian cells or tissues. Alternatively, the receptor protein of the present invention or salts thereof may also be manufactured by culturing a transformant containing DNA encoding the receptor protein of the present invention, as will be later described. Furthermore the receptor protein of the present invention or salts thereof may also be manufactured by known methods for synthesizing proteins.

Finally, the invention provides two specific proteins, one being truncated or deleted, and the other being mutated. The invention also provides the polynucleotides (purified) encoding said proteins, a vector comprising said polynucleotide and a host cell comprising the vector.

Also within the ambit of the invention is the antisense nucleic acid, as well as the gene therapy using the above GPR54 receptor.

The G protein coupled receptor may be used not only for administration of antagonists or agonists of the receptor, but also for gene therapy by transfer of the receptor gene into the body (or certain specific organs such as the hypophysis) or by transfer of the antisense nucleic acid to the receptor gene.

Antisense nucleic acids that can inhibit replication or expression of the GPR54 receptor protein gene can inhibit RNA synthesis or the function of RNA, or can regulate/control the expression of the receptor protein gene via the interaction with RNAs associated with the receptor protein. Antisense nucleic acids are useful for regulating and controlling the expression of the receptor protein gene in vivo and in vitro, and are also useful for the treatment and diagnosis of the diseases disclosed above.

Technologies related to such antisense RNAs and gene therapies are known to the skilled man.

### EXAMPLES

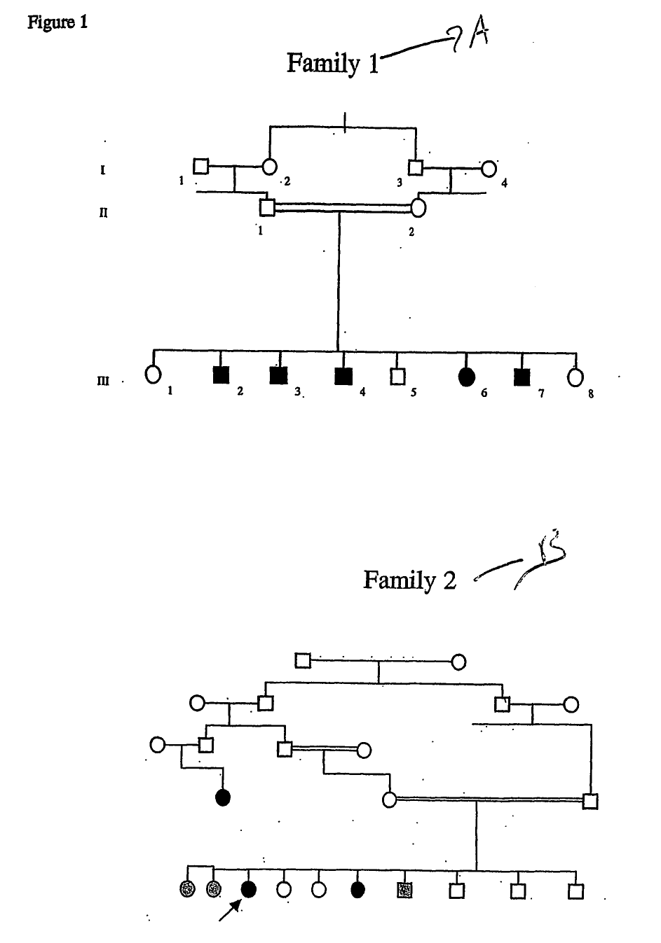

A consanguineous family (family 1) with 5 affected sibs was investigated (see FIG. 1). The propositus was a 20-year old male referred for impuberism. He had typical signs of hypogonadism with small testes (28×17 mm), sparse pubic hair (P3) and a penis of 7 cm. His bone age was retarded at 15.0 years. He had a normal sense of smell and showed no abnormal eye movements, no colour blindness and no renal or cranio-facial abnormalities. His weight and height were 54 kgs and 152 cm respectively. Three brothers presented similar clinical signs. A sister had a partial hypogonadism. At 16, she had partial breast development and she reported a single episode of uterine bleeding. Hormone assays (Table 1) showed low plasma testosterone in boys and low plasma oestradiol in the sister accompanied by low plasma gonadotropin levels. All sibs had a partial or a blunted response to GnRH (100 82 g IV). One other brother and two other sisters had a normal pubertal development. The parents were first cousins and have had normal pubertal development. Table 1 below gives the hormonal status of the affected patients of family 1

| | | | Plasma | Plasma | Plasma | Plasma | GnRH | |
|---|---|---|---|---|---|---|---|---|
| | | Bone | Testosterone | Oestradiol | LH | FSH | test | |
| Patient | Age | age | (ng/dl) | (pg/ml) | (mU/ml) | (mU/ml) | LH | FSH |
| III.2 | 21 | 15 | 26 | — | 1.5 | 0.5 | 3.6 | 1.7 |
| III.3 | 20 | 15 | 19 | — | 1.5 | 0.5 | 1.4 | 1.5 |
| III.4 | 19 | — | 5 | — | 1.1 | 4.1 | 1.9 | 4.1 |
| III.6 | 18 | — | — | 17 | 2.0 | 3.4 | 11.8 | 6.4 |
| III.7 | 14 | 11 | 5 | — | 2.6 | 1.8 | 3.4 | 2.6 |

The chronological age and the bone age are indicated. Normal values: (males) LH, 1.0-5.0 IU/ml, FSH 0.9-5.7 IU/ml), Testosterone 260-690 ng/dl; (Females) LH, 1.1-5.4 IU/ml, FSH 2.3-6.0 IU/ml, Oestradiol (early follicular phase) 25-90 μg/ml. The GnRH test was performed by intra venous administration of 100 μg of GnRH. The highest values observed for plasma LH and FSH are reported.

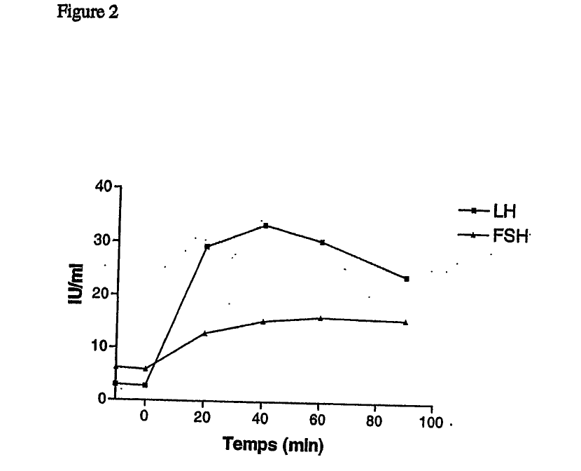

The second family (family 2) was a consanguineous family originated from Kurdistan. The propositus was a 27 years old woman referred for primary amenorrhea. She had a normal breast and pubic hair development. She had a normal sense of smell. Ultrasonography showed a small uterus with thin infantile endometrium. Ovaries were small with several small immature follicles. Plasma oestradiol is low accompanied by normal plasma gonadotropins. All other anterior pituitary hormone plasma levels were in normal range. The GnRH test performed with 100 μg/IV showed a normal response for the FSH and an explosive response for LH (see FIG. 2). LH pulsatility showed low amplitude peaks but normal frequency. A pulsatile GnRH pump administration led to a normal pregnancy.

For both family sibs, genomic DNA was isolated from peripheral lymphocytes following standard methods. The 5 exons of the GPR54 gene were amplified by PCR with 20 to 100 ng of genomic DNA. The following primers were used:

| | | |
|---|---|---|
| Exon 1: | Forward: | GGGCGGCCGGGAGGAGGA |
| | Reverse: | CCGGGACGGCAGCAGGTG |
| Exon 2: | Forward: | GCCCAGCGCCCGCGCATC |
| | Reverse: | GTCCCCAAGTGCGCCCTCTC |
| Exon 3: | Forward: | CAGGCTCCCAACCGCGCAG |
| | Reverse: | CGTGTCCGCCTTCTCCCGTG |
| Exon 4: | Forward: | CTTCATCCTGGCTTGTGGCAC |
| | Reverse: | CTTGCTGTCCTCCCACCCAC |
| Exon 5: | Forward: | GCCTTTCGTCTAACCACCTTC |
| | Reverse: | GGAGCCGCTCGGATTCCCAC |

Amplification was performed for 30 cycles with Yellow Taq (Eurogentec) in 1.5 mM MgCl2, with 0.1 μM of each primer and 5% DMSO. The annealing temperatures were of 600 for exons 1, 3, 4, 5 and of 660 for exon 2. The PCR products were directly sequenced with BigDye dideoxyterminator cycle sequencing kits and the 3100 sequencer (Applied Biosystems) using the same primers. To genotype all members of the family, the PCR products of exon 5 were analyzed by electrophoresis in 2% agarose gel.

Upon study of the GPR54 gene, it was observed in affected individuals a homozygous deletion of 155 base pairs lying between intron 4 (nucleotide −13 when numbering from the 3′ end of the intron 4) and exon 5 (nucleotide 142 of the exon 5, corresponding to nucleotide 880 of the cDNA). The deletion reported in family 1 removes the splicing acceptor site of intron 4-exon 5 junction. It thus leads to the absence of the normal protein sequence downstream from residue 247 (FIG. 3). The deleted receptor is truncated within the third intracellular loop thus lacking transmembrane domains 6 and 7 (FIG. 3). All affected patients were homozygous for this deletion. Both parents as well as unaffected sib III.5 were heterozygous. Unaffected sib III.1 was homozygous for the wild type sequence. The deletion was absent in 50 control subjects.

In family 2 showing a recessive transmission, a homozygous point mutation was found within exon 1. This mutation substituted a proline for a leucine (L102P) at the N-terminal extremity of the first extracellular loop.

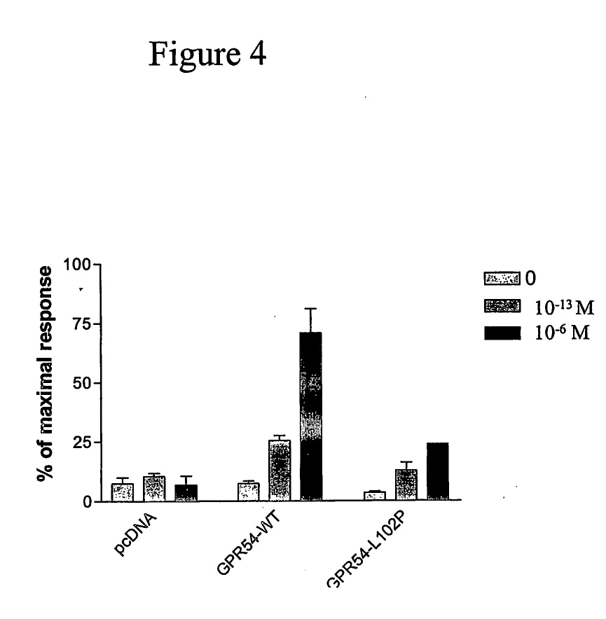

The L102P mutation was reproduced by PCR in-vitro mutagenesis and sub-cloned within a human GPR54 expressing vector (pGPR54_L1O2P). The functional characterisation of the L102P mutated receptor was performed after transient transfection of the wild type and mutated L102P plasmid within HEK293T cells. The activation of G-protein activated phospholipase C-β, was evaluated after stimulation by different concentrations of Kiss1 peptide (decapeptide 45-54, or fragment 45-54 being a functional truncation of Kiss-1). Inositol phosphate accumulation was measured after [H3] inositol cell labelling and ion exchange chromatography purification. Dose response clearly showed that substitution of leucine 102 by a proline inactivates phospholipase C stimulation in HEK293T cells (see FIG. 4). L102P mutation is thus a loss of function mutation of GPR54. This demonstrates that gonadotropic deficiency observed in family 2 is also due to the inactivation of GPR54 function by L102P mutation.

GPR54 is thus involved in the regulation of the LH and FSH synthesis. There is strong evidence that the Kiss-1 peptide is the GPR54 ligand involved in this physiological process as evidenced by the characterisation of a genetic abnormality in a hypogonadotropic hypogonadism patient born from first cousin parents. A duplication in tandem of 20 nucleotides localized at the 3′ end of exon 3 of the human kiss-1 gene has been found. This duplication does not change the C-terminal end of the peptide but is likely to disturb the mRNA stability or translation. This duplication was not found in 400 chromosomes which demonstrates that it is not a polymorphism.

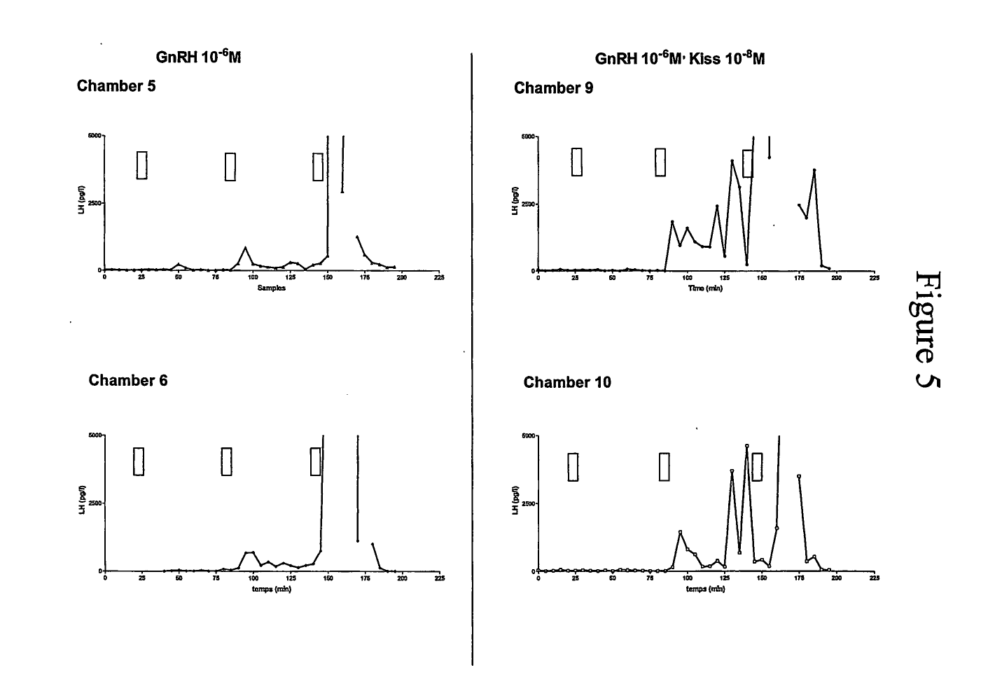

Physiological experiments have confirmed that the Kiss-1 peptide is the GPR54 ligand. GPR54 is expressed within the pituitary. LH plasmatic pulsatility measured in the propositus of family 2 has suggested that GPR54 inactivation defect occurs at the pituitary level. An approach using rat perifused pituitaries was used to study the effects of Kiss-1 stimulation on pituitary LH secretion. Rat pituitaries were in-vitro perifused with different concentrations of Kiss-1, GnRH or with a combination of Kiss-1 with GnRH (see FIG. 5). LH levels were measured at 5 minute intervals. A single peak was observed immediately after the injection of the GnRH and then the levels returned to the base line level. This stimulation was marked after the second GnRH injection as expected (see chamber 5 and 6). Such priming effect was already described for the GnRH. After stimulation with both GnRH and Kiss-1 (peptide 45-54 or fragment 45-54, a functional truncated variant of Kiss-1), one peak was observed immediately as described for the GnRH alone. However, the LH levels did not return to the basal line level (see chambers 9 and 10). The total LH output (area under the curve) was clearly increased in chambers stimulated by both agonists when compared with GnRH alone (chambers 5 and 6). No stimulation was observed with Kiss-1 alone, whatever the concentrations used (10⁻¹⁰, 10⁻⁸, 10⁻⁶). This demonstrates that Kiss-1 extend the effect of the GnRH on the LH synthesis stimulation by pituitaries.

It has thus been demonstrated that Kiss-1 positively modulates the GnRH effect on LH synthesis stimulation. Kiss-1 may be classified as a strong modulator of the GnRH effect as the functional integrity of its receptor GPR54 is required for a normal activation of the gonadotropic axis. The perifused pituitaries approach shows that Kiss-1 directly acts on pituitary cells. These results provide evidence that agonists or antagonists of GPR54 can modulate LH synthesis stimulation by the GnRH.

In compositions comprising GnRH and suitable agonists or antagonists of GPR54, suitable ratios of GnRH to the agonist/antagonist range between 10:1 to 1000:1 in Molar concentration.

1-28. (canceled)

29. A composition comprising GnRH and Kiss-1 peptide, Kiss-1 peptide fragments, kisspeptins, or salt thereof.

30. A composition according to claim 29, wherein the Kiss-1 peptide fragment is the fragment 45-54 of Kiss-1.

31. A composition according to claims 29 or 30, wherein the ratio of the GnRH to Kiss-1 peptide, Kiss-1 peptide fragments, kisspeptins, or salt thereof, is in the range 10:1 to 1000:1 in Molar concentration.

32. The composition according to any of one of the claims 29 to 31 for its therapeutic use in the treatment of a gonadotropin related reproductive disorder.

33. The composition according to claim 32 wherein the therapeutic use is the treatment of LH and/or FSH related disorders.

34. The composition according to claim 32 wherein the therapeutic use is the stimulation of ovulation.

35. The composition according to claim 32 wherein the therapeutic use is to positively modulate LH synthesis.

36. Kiss-1 peptide, Kiss-1 peptide fragments, kisspeptins, or salt thereof for the use as an addition to a treatment for the stimulation of ovulation by GnRH.

37. Kiss-1 peptide, Kiss-1 peptide fragments, kisspeptins, or salt thereof for the use according to claim 36 wherein the Kiss-1 peptide, Kiss-1 peptide fragments, kisspeptins, or salt thereof positively modulates the GnRH effect on LH synthesis stimulation.

38. A Kiss-1 peptide fragment for the use according to claims 36 or 37, wherein the Kiss-1 peptide fragments is the fragment 45-54 of Kiss-1.

39. A Kiss-1 peptide, Kiss-1 peptide fragments, kisspeptins, or salt thereof for the use according to any one of the claims 36 to 38, wherein the ratio of the GnRH to Kiss-1 peptide, Kiss-1 peptide fragments, kisspeptins, or salt thereof, is in the range 10:1 to 1000:1 in Molar concentration.


SEQUENCE LISTING (3 sequences)

SEQ ID NO: 1 - DNA, 1197 bp, Homo sapiens
  CDS (1)..(1197)
  gene (1)..(1197): GPR54 gene
atgcacaccgtggctacgtccggacccaacgcgtcctggggggcaccggccaacgcctccggctgcccgggctgtggcgccaacgcctcggacggcccagtcccttcgccgcgggccgtggacgcctggctcgtgccgctcttcttcgcggcgctgatgctgctgggcctggtggggaactcgctggtcatctacgtcatctgccgccacaagccgatgcggaccgtgaccaacttctacatcgccaacctggcggccacggacgtgaccttcctcctgtgctgcgtccccttcacggccctgctgtacccgctgcccggctgggtgctgggcgacttcatgtgcaagttcgtcaactacatccagcaggtctcggtgcaggccacgtgtgccactctgaccgccatgagtgtggaccgctggtacgtgacggtgttcccgttgcgcgccctgcaccgccgcacgccccgcctggcgctggctgtcagcctcagcatctgggtaggctctgcggcggtgtctgcgccggtgctcgccctgcaccgcctgtcacccgggccgcgcgcctactgcagtgaggccttccccagccgcgccctggagcgcgccttcgcactgtacaacctgctggcgctgtacctgctgccgctgctcgccacctgcgcctgctatgcggccatgctgcgccacctgggccgggtcgccgtgcgccccgcgcccgccgatagcgccctgcaggggcaggtgctggcagagcgcgcaggcgccgtgcgggccaaggtctcgcggctggtggcggccgtggtcctgctcttcgccgcctgctggggccccatccagctgttcctggtgctgcaggcgctgggccccgcgggctcctggcacccacgcagctacgccgcctacgcgcttaagacctgggctcactgcatgtcctacagcaactccgcgctgaacccgctgctctacgccttcctgggctcgcacttccgacaggccttccgccgcgtctgcccctgcgcgccgcgccgcccccgccgcccccgccggcccggaccctcggaccccgcagccccacacgcggagctgctccgcctggggtcccacccggcccccgccagggcgcagaagccagggagcagtgggctggccgcgcgcgggctgtgcgtcctgggggaggacaacgcccctctctga

SEQ ID NO: 2 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELLRLGSHPAPARAQKPGSSGLAARGLCVLGEDNAPL

SEQ ID NO: 3 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELHRLGSHPAPARAQKPGSSGLAARGLCVLGEDNAPL